(19) United States
(12) Patent Application Publication
(10) Pub. No.: US 2012/0103341 A1
(43) Pub. Date: May 3, 2012

(54) TRACHEAL TUBE WITH CONNECTOR INSERT

(75) Inventor: Mark Behlmaier, Erie, CO (US)

(73) Assignee: Nellcor Puritan Bennett LLC, Boulder, CO (US)

(21) Appl. No.: 12/916,297

(22) Filed: Oct. 29, 2010

(51) Int. Cl. A61M 16/04; B23P 17/04

(52) U.S. Cl. 128/207.14; 29/428

(57) ABSTRACT

A tracheal tube assembly includes a connector body, a cannula extending from the connector body, and an insert that provides rigidity to the connector body and retains the cannula in the connector body. The insert has a proximal surface having a first outside diameter substantially the same size as a second outside diameter of the connector body. The cannula has a tapered upper end that fits between conforming tapered sections of the connector body inner surface and the insert. The insert may include features to prevent rotation of the cannula as well as rotation of the insert in the connector body. Features of the insert and connector body may also interface to mechanically retain the insert in the connector body. A fastening component may be provided to aid in the fastening of the insert and cannula to the connector body. Different sizes of cannula may be accommodated by different inserts, while using the same or different connector bodies.

### BACKGROUND

The present disclosure relates to a tracheal tube, and more particularly to a tracheal tube having a connector insert for rigidifying a connection and securing a cannula to the connector.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide range of applications exist for artificial ventilation, which may call for the use of tubes that are inserted into a patient. Such tubes may include endotracheal tubes, tracheostomy tubes, and so forth. In the former case, the tubes are typically inserted through the mouth and into the trachea. In the latter, the tubes are often inserted into an opening formed in the neck and trachea of the patient. In both cases, the tubes may be used for artificial ventilation or for assisting patient ventilation. They are typically designed to interface with standard connectors that are located at the end of a ventilation hose assembly which itself may be connected to a ventilator.

Current designs for such tubes may allow for easy connection to an upper connector, but may have various structures, some quite complex, for conveying air between the connector and a cannula that extends into the patient. In some cases, a soft plastic or rubber is used for the connector, providing a flexible seal with the interfacing ventilation assembly, although such soft materials may collapse or deform when pressed into the mating connector element. Moreover, difficulties exist in the mounting of the cannula in such devices, which must interface with the connector portion to provide the desired airflow path. The sizes of such cannulas may vary substantially, depending upon the anatomy of the patient, the age of a patient, and so forth. For example, the inner diameter of cannulas for pediatric and neonatal patients may vary between about 2.5 mm and 6.5 mm. Larger sizes may be provided, but it would be desirable to have a uniform system of attachment between the cannula and the connector independent of the size.

### SUMMARY

Provided herein is a novel arrangement for a tracheal tube designed to respond to such needs. In accordance with one embodiment, an end connector is provided having a generally annular body. A cannula has an upper end that is disposed in the annular body of the connector. An insert is disposed in the annular body of the connector and has a tubular lower extremity that contacts an upper end of the cannula to retain the cannula lodged within the annular body. The insert includes a proximal surface having a flange-like portion with an outside diameter substantially the same size as an outside diameter of the annular body.

In a similar arrangement, a tracheal tube may include an end connector that has a generally annular body with an outer surface dimensioned to a standard connector size. A cannula has an upper end. An insert is disposed in the annular body and has a tubular lower extremity that contacts the upper end of the cannula. A fastening component is disposed on a rear end of the annular body and contacts the insert. The cannula is compressed between the insert and the fastening component to retain the cannula.

Also provided is a method for making a tracheal tube. The method includes inserting an insert into a cannula. The insert and cannula are inserted into an upper opening of the an annular body of a connector to expand and compress an upper end of the cannula against an inner surface of a fastening component or the connector to retain the cannula in the connector. The insert includes a proximal surface having a first outside diameter substantially the same size as a second outside diameter of the annular body. It should be noted that an insert having a similar structure and method of manufacture is disclosed in patent application Ser. No. 12/788,569 to Paul Waldron et al., filed on May 27, 2010, and entitled “Tracheal Tube with Connector Insert,” which is hereby incorporated by reference as if fully set forth herein.

### BRIEF DESCRIPTION OF THE DRAWINGS

Advantages of the disclosed techniques may become apparent upon reading the following detailed description and upon reference to the drawings in which:

FIG. 1 is a perspective view of a tracheal tube in accordance with aspects of the present disclosure;

FIG. 2 is an exploded view of the tracheal tube of FIG. 1;

FIG. 3 is a sectional view of the same arrangement, illustrating internal features of the connector body, the insert, and the cannula when the three are joined in the completed tracheal tube; and

FIG. 4 is a sectional view of another aspect of the insert, illustrating internal features of the insert and a fastening component.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

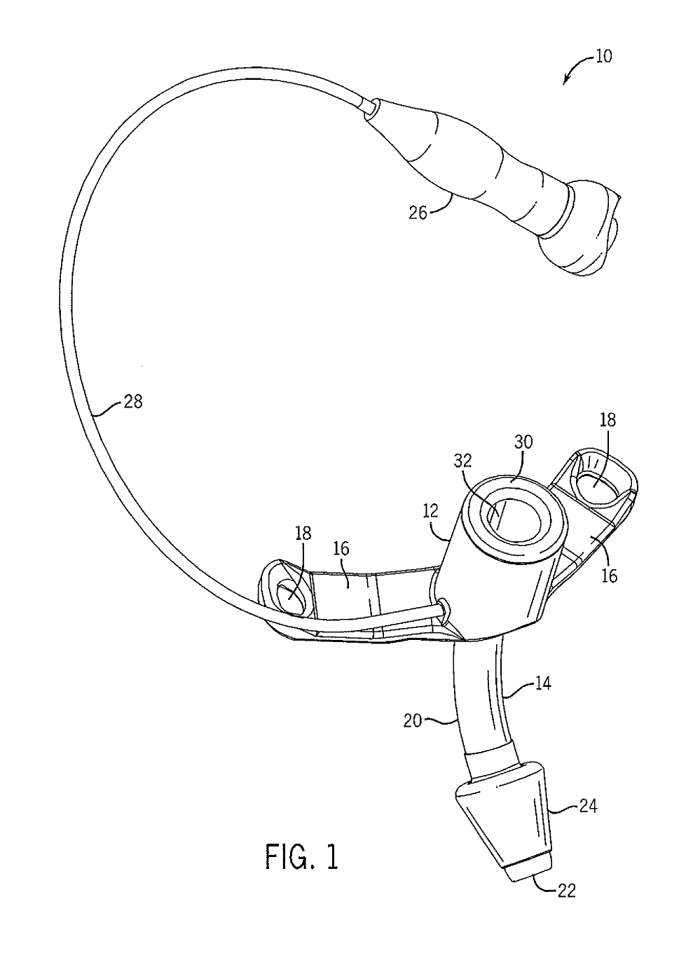

FIG. 1 illustrates an exemplary tracheal tube in accordance with the present disclosure, designated generally by reference numeral M. In the illustrated embodiment, the tracheal tube is designed as a tracheostomy tube, although the present assembly techniques could be used in other tubes, such as endotracheal tubes and so forth. In the illustrated embodiment, the tracheal tube 10 includes an end connector 12 designed to be attached to an artificial ventilation system. Various sizes of such connectors are available, and the connector may be dimensioned to accommodate any one of these sizes. In the illustrated embodiment, the connector has an outer diameter of approximately 15 mm to conform to ISO standard dimensions of mating connectors. A cannula 14 extends from a lower end of the connector and is designed for insertion into the airway of a patient. Moreover, side flanges 16 extend from the connector for facilitating securement of the device to a patient. In the case of the tracheal tube shown, the flanges 16 may terminate in apertures 18 through which straps or other attachments devices can be inserted. The tracheal tube may be held in place on the neck of a patient by such straps. It should be noted that certain arrangements that incorporate the present teachings, such as endotracheal tubes, may not be provided with flanges.

In the illustrated embodiment, the cannula 14 is a hollow tube that can direct air or other ventilation gasses into and out of a patient. To conform more aptly to the patient anatomy, a curved section 20 may be provided as shown. The curved section ends in a lower or distal tip 22 which will be lodged in the patient during use. In certain embodiments, the distal tip 22 may include a beveled edge enabling a smoother insertion of the cannula 14 into the patient's trachea. The illustrated cannula 14 includes an inflatable cuff 24 designed to seal the patient's airway. The cuff 24 may be connected to a cuff inflator valve 26 through an inflation lumen 28. The cuff inflator valve 26 may deliver a gas, such as air, through the inflation lumen 28 and into the cuff 24, thus inflating the cuff 24. The cuff 24, when inflated, will expand radially around the cannula 14 and seal the patient's airway. By using one or more cuffs 24 to seal the patient's airway, substances may flow only through the cannula 14 (or other medical device), allowing better control over the type and amount of substances flowing into and out of the patient.

The end connector 12 further includes an insert 30 which is disposed inside the connector body as described more fully below. As also described below, the insert serves to rigidify the connector body and to retain the cannula within the connector body. The insert may also include a top surface. Moreover, the insert may assist in preventing rotation of the cannula within the connector body. An air passageway 32 is formed through the insert 30 and extends through the cannula such that, when coupled to appropriate ventilation devices, air or other gasses may be freely exchanged between the upper or proximate end of the connector and the distal end 22 of the cannula 14.

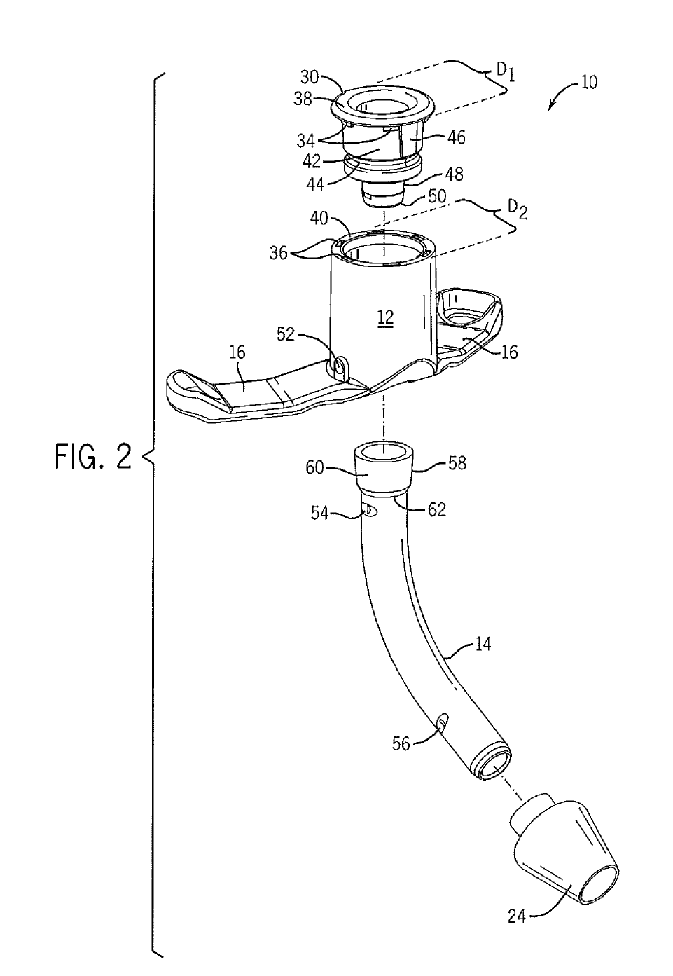

FIG. 2 is an exploded view of embodiments of components of the tracheal tube 10 that may include features, such as engageable teeth 34, capable of securing the insert 30 into the end connector 12. The engageable teeth 34 may be inserted into apertures or holes 36 of the end connector 12, and may aid in preventing the rotation of the insert 30 within the connector body. It is to be understood that while the depicted embodiment illustrates rectangular teeth, other embodiments may include pegs, triangular teeth, curved teeth, and so forth. Indeed, in another embodiment, the teeth 34 may be replaced with, for example, a full or sectional annular ridge. The annular ridge may then engage a conforming annular channel on the connector 12.

Moreover, the insert 30 may include a proximal flange forming a surface 38 (i.e., “top” surface) that may substantially surround a proximal end 40 of the connector 12. This flange may have substantially the same radial dimension as the connector body. That is, in one embodiment, the top surface 38 may include an outer diameter D₁ slightly smaller to an outer diameter D₂ of the proximal end 40. For example, D₁ may be approximately between 1/1,000 in. to 1/50 in. smaller than D₂. The slight size difference between D₁ and D₂ prevents D₁ from creating an interference fit with a corresponding ventilator connector (e.g., female end connector) that may be coupled to the end connector 12. The end connector body may create an interference fit suitable for securing, for example, the female end connector to the connector 12. In another embodiment, the outer diameter D₁ may be approximately equal to the outer diameter D₂. By “covering” the proximal end 40, the top surface 38 may reduce or eliminate the number of interstices (e.g., spaces or gaps) included in the tracheal tube 10, thus reducing the locations that may harbor bacteria. The figure is also illustrative of how the various components of the tracheal tube 10 may be assembled or manufactured.

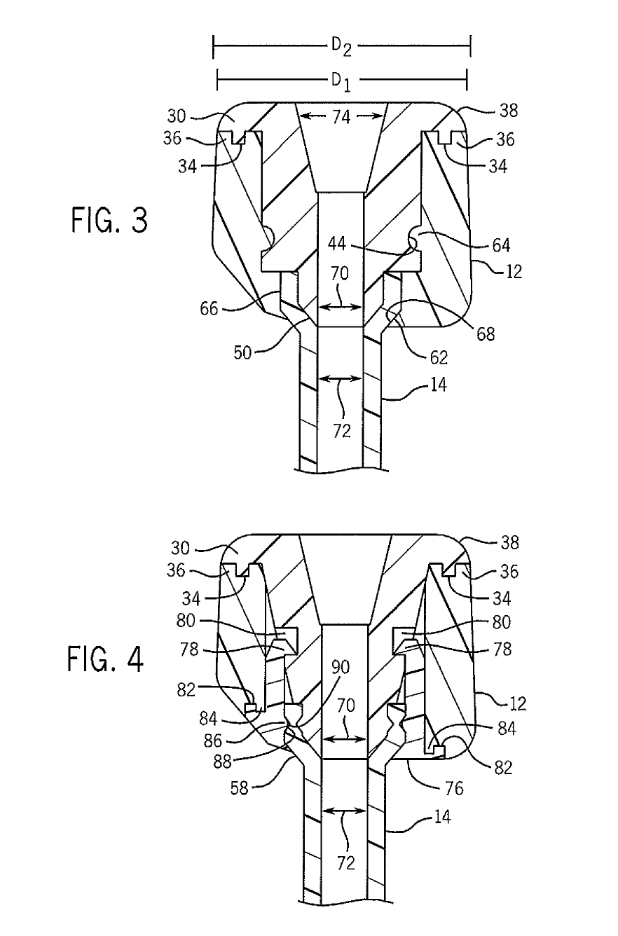

The insert 30 includes a body 42. In certain embodiments, the body 42 may be a generally tapered body 42. In other embodiments, the body 42 may be a generally cylindrical body 42. In the particular embodiment illustrated, a groove 44 is provided near the lower end of the insert, and this groove 44 will interface with a conforming feature of the connector body when inserted, as also described below. Moreover, a flat or other key structure 46 is provided that also aids in preventing rotation of the insert within the connector body. Also visible in FIG. 2 is a lower extremity 48 of the insert. As described with particular reference to FIGS. 3 and 4, this lower extremity 48 is configured to conform to an upper end of the cannula 14. The lower extremity 48, in turn, has a tapered portion 50 that interfaces with the cannula as described below. In certain embodiments, the inner surface of the end connector body may be coated with a glue so as to securely couple the cannula 14 and/or insert 30 to the end connector body.

The connector 12 includes an aperture 52 suitable for enabling the insertion of the inflation lumen 28 (shown in FIG. 1). A fluid such as air may be delivered to the cuff 24, for example, through the inflation lumen 28, via an internal cannula lumen having openings 54 and 56. Accordingly, the cuff 24 may be inflated to comformably seal the patient's airway. The cannula itself has an upper end 58 which forms a tapered upper section 60 configured to fit against the inner surface of the connector body as described below. A lower tapered section 62 also contacts and interfaces with the inner surface of the connector body to retain the cannula in place within the connector. The four components 12, 14, 24 and 30, will typically be formed separately. The connector 12 and insert 30 will typically be molded, while the cannula 14 may be made by an extrusion process. Other processes may, of course, be employed where desired and appropriate.

In a presently contemplated embodiment, the connector 12 is made of a soft polyvinylchloride or other plastic. The soft material of the connector allows for easy gripping and a good contact fit with the mating connector part when the tube is connected to a ventilation system. The softer material also allows for comfort against the patient's neck. The flanges 16 may also be molded with the body of the connector 12, or these could be added in a separate operation. In a presently contemplated embodiment, the flanges 16 are co-molded or over-molded with the connector body. The cannula 14 may also be made of a plastic material, such as soft polyvinylchloride, polyurethane, thermoplastic elastomers, or other plastics. The insert 30 may be made of a harder material than the connector body, such as a hard polyvinylchloride, a polycarbonate plastic, ABS, or any suitable material or a combination of materials. Where the insert is harder than the soft connector body, it provides rigidity to a connector body and resists forces that might tend to collapse the connector body, such as from mating connectors, and so forth. The more rigid structure also provides a good surface to which the cannula may be bonded, and that supports the inner diameter of the cannula.

In one embodiment, the assembly may be performed by first inserting the insert 30 into the cannula 14. For example, the lower extremity 48 may be concentrically or co-axially inserted into the upper end 58 by applying a mechanical force. Various fastening techniques may be used to secure the insert 30 to the cannula 14. In one example, a solvent bonding or solvent welding is used. In this example, a solvent is used to coat the mating surfaces of the insert 30 and cannula 14 to fasten the two components. An interference or compression fit and the evaporation and/or thermal activation of the solvent may result in a strong bonding of the insert 30 to the cannula 14. In this example, the solvent bonding may reduce undesirable adhesive or glue residue. In another example, an adhesive or glue may be used. The adhesive may be applied to the outer surface of the lower extremity 48 and/or the inner surface of the upper end 58, thus securely fastening the two components to each other. For example, a cyanoacrylate glue may be used to fasten the two components 48 and 58 to each other.

Both the cannula 14 and the insert 30 may then be inserted into the connector 12, such that the cannula lower end extends through the connector 12 and the cannula seats within the connector as described below. During insertion, the teeth 34 are aligned with the apertures 36 and the insert 30 is aligned with the connector inner surface and pressed into place. In the present embodiment, the retention features of the insert 30, such as teeth 34 and groove 44, cooperating with those of the connector 12, prevent the insert from being easily removed from the connector. Although mechanical features are built into the connector and insert in the embodiment illustrated, such mechanical features may be complimented by various bonding agents and/or adhesives. For example, the solvent bonding or solvent welding technique described above may be used to securely bond the insert (and cannula 14), to the connector 12. In certain embodiments, the insert 30 and connector body may be co-molded or over-molded. In another embodiment, a fastening component may then be inserted through the bottom of the connector 12, as described in more detail below with respect to FIG. 4, and fastened in place. The cuff 24 may then be disposed in the distal end of the cannula 14, and aligned over the opening 56. Both the fastening component and the cuff 24 may then be secured through the use of various bonding agents and/or adhesives. It should be noted that the assembly may proceed in different orders (e.g., by insertion of the cannula 14 in the connector with or separately from the insert 30), depending upon the particular configuration of the components, the nature of the retaining features of each, and the type of processes used for formation and assembly (e.g., solvent bonding, overmolding, etc.).

FIG. 3 illustrates the three components 12, 14, and 30 of the tracheal tube in section. As described above, when assembled, the cannula 14 is lodged within the connector body, and retained in place by the insert 30. Moreover, the flat structure 46 illustrated in FIG. 2 cooperates with a similar surface of the connector body to prevent rotation of the insert within the connector body. The flat structure 46 may also be used as an assembly aid to facilitate alignment of the insert 30 with respect to the end connector 12 body. Further, the flat structure 46 may be used as a “keying” feature for repeatable positioning on a semi-automated assembly fixture. In the arrangement shown in FIG. 3, moreover, an inner protrusion 64 of the connector body enters into groove 44 of the insert 30 to mechanically retain the insert 30 within the connector body. Likewise, the teeth 34 enter the apertures 36 of the insert 30 to aid in securing the insert 30 to the connector 12 and in maintaining the alignment of the insert 30 co-axially with respect to the connector 12. Here again, adhesives and bonding agents may also be employed to retain these components in the assembled positions shown in FIG. 3. Still further, the lower tapered section 50 of the insert (see FIG. 2) is configured to conform to a similar upper tapered inner wall section 66 of the connector body. The lower tapered section 62 of the cannula upper end (see FIG. 2) similarly conforms generally to a lower tapered inner wall section 68 of the connector body. Thus, the insert 30, which fits within the upper end of the cannula 14, tends to expand or compress the upper end of the cannula 14 slightly against the inner surface of the connector body. This cooperation retains the cannula 14 within the connector body, and prevents rotation of the cannula 14 with the connector body.

It should be noted that the sizes of these components may be adapted to conform to various standard sizes of tracheal tubes. For example, in tubes used for pediatric and neonatal patients, an inner diameter of the cannula may vary between 2.5 and 6.5 mm. Other sizes, could, of course, be accommodated. It should also be noted that, as shown in FIG. 3, the outer diameter D₁ of the top surface 38 may be slightly smaller than the outer diameter D₂ of the connector body. Additionally, the inner diameter of the lower extremity of the insert 30, indicated by reference numeral 70, will generally conform to the inner diameter 72 of the cannula. This arrangement allows for the easy passage of air or other ventilation gasses without creating an obstruction either in the connector or the cannula. The upper end of the opening in the insert, indicated by reference numeral 74, preferably expands to allow for the channeling of air or ventilation gasses easily into the assembly. It should also be noted that a range of sizes of inserts may be accommodated for the same external dimension of the connector body. Thus, various sizes of inserts 30 may be designed to interface with various sizes of cannulas. This may be done while maintaining the configuration and even the size of the connector body the same. Thus, the same connector body may be used with different inserts and cannula sizes to obtain a family of tracheal tubes. It is to be understood that, in other embodiments, the end connector body may be modified to accommodate cannulas of different sizes.

FIG. 4 illustrates the three components 12, 14, and 30 of the tracheal tube in section, with an additional fastening component 76. The fastening component 76 may enable a faster assembly of the tracheal tube 10 by using techniques such as a snap fit or hook 78. As mentioned above, one assembly example has the insert 30 inserted into the cannula 14. The insert 30 and the cannula 14 may then be inserted into the connector 12, such that the teeth 34 engage the apertures 36 of the connector 12. In the illustrated embodiment, the fastening component 76 may then be inserted from the rear of the end connector body until the hook 78 engages a groove 80 of the insert 30. The hook 78 may be an annular hook circumferentially disposed on an inside wall of the fastening component 76. Likewise, the groove 80 may be circumferentially disposed on an exterior wall of the insert 30. Additionally, teeth 82 of the fastening component 76 may be inserted into apertures 84 of the connector 12 to aid in securing the fastening component 76 to the connector 12.

In this embodiment, the fastening component 76 may also aid in the lodging and compression of the upper end 58 of the cannula 14 against the insert 30 by providing for a more rigid structure than the connector body. Lodging the upper end 58 of the cannula 14 between two more rigid structures (e.g., insert 30 and fastening component 76) thus increases the compressive force experienced by the upper end 58. Moreover, the fastening component 76 may include a protrusion 86 comformably shaped to fit into a groove 88 of the upper end 90, which may be formed on the through compression between the insert 30 and the fastening component 76. That is, the groove 88 may not manufactured on the cannula 14 but may be the result of compressive forces. Such an improved fastening allows for a stronger coupling with the cannula 14 and prevents rotation of the cannula 14 with the connector body. Moreover, the speed and efficiency of assembly of the tracheal tube 10 may be improved by “snapping” the fastening component 76 into the rear of the connector 12 after insertion of the insert 30 and cannula 14. That is, once the insert 30 and the cannula 14 are inserted into the connector 12, the fastening component may be inserted such that the hook 78 “snaps” into the groove 80. Further, the use of the fastening component 76 may reduce the need for adhesives and/or bonding agents by using mechanical fastening techniques.

What is claimed is:

1. A tracheal tube comprising:
a connector having a generally annular body;
a cannula having an upper end disposed in the annular body; and
an insert disposed in the annular body and having a tubular lower extremity contacting the upper end of the cannula to retain the cannula lodged within the annular body, wherein the insert comprises a proximal flange surface having a first outside diameter substantially the same size as a second outside diameter of the annular body.

2. The tracheal tube of claim 1, wherein the first outside diameter is approximately between 1/50 inch and 1/1,000 inch smaller than the second outside diameter.

3. The tracheal tube of claim 1, wherein the tubular lower extremity of the insert has an inner diameter approximately equal to an inner diameter of the cannula.

4. The tracheal tube of claim 1, wherein the annular body comprises a retaining feature that interfaces with the insert to prevent removal of the insert from the annular body.

5. The tracheal tube of claim 1, comprising a fastening component configured to aid in fastening the insert and cannula to the annular body, wherein the fastening component is disposed in a rear end of the annular body.

6. The tracheal tube of claim 1, wherein the lower extremity of the insert is tapered to expand the upper end of the cannula towards an inner surface of the connector.

7. The tracheal tube of claim 1, wherein the annular body is made of an elastomeric material, and the insert is made of a material harder than the elastomeric material of the annular body.

8. The tracheal tube of claim 1, wherein the insert has an inner surface tapered from a first inner diameter within the insert to a second larger inner diameter at an upper end of the insert.

9. A tracheal tube comprising:
a connector having a generally annular body with an outer surface dimensioned to a standard connector size;
a cannula having an upper end;
an insert disposed in the annular body and having a tubular lower extremity contacting the upper end of the cannula; and
a fastening component disposed on a rear end of the annular body and contacting the insert, wherein the cannula is compressed between the insert and the fastening component to retain the cannula.

10. The tracheal tube of claim 9, wherein the fastening component comprises a hook and wherein the insert comprises a groove suitable for engaging the hook.

11. The tracheal tube of claim 9, wherein at least a portion of the lower extremity of the insert is tapered to expand the upper end of the cannula towards the inner surface of the annular body.

12. The tracheal tube of claim 9, wherein the insert comprises a proximal surface having a first outside diameter substantially the same size as a second outside diameter of the annular body.

13. The tracheal tube of claim 9, wherein the annular body comprises an anti-rotation feature that interfaces with the insert to prevent rotation of the insert within the annular body.

14. The tracheal tube of claim 9, wherein the annular body comprises a retaining feature that interfaces with the insert to prevent removal of the insert from the annular body.

15. The tracheal tube of claim 9, wherein the annular body is made of an elastomeric material, and both the insert and the fastening component are made of a material harder than the elastomeric material of the annular body.

16. A method for making a tracheal tube, comprising:
inserting an insert into a cannula; and
inserting the insert and cannula into an upper opening of an annular body of a connector to expand and compress an upper end of the cannula against an inner surface of a fastening component or the connector to retain the cannula in the connector, wherein the insert comprises a proximal flange surface having a first outside diameter substantially the same size as a second outside diameter of the annular body.

17. The method of claim 16, wherein the first outside diameter is approximately between 1/50 inch and 1/1,000 inch smaller than the second outside diameter.

18. The method of claim 16, wherein the annular body is made of an elastomeric material, and both the insert and the fastening component are made of a material harder than the elastomeric material of the annular body.

19. The method of claim 16, comprising engaging anti-rotation features of the connector and the insert to prevent rotation of the insert in the connector.

20. The method of claim 16, comprising engaging retaining features of the fastening component and the insert to prevent removal of the insert from the connector.
